(19) United States
(12) Patent Application Publication
(10) Pub. No.: US 2006/0200234 A1
(43) Pub. Date: Sep. 7, 2006

(54) Endovascular aneurysm treatment device and delivery system

(76) Inventor: Richard Allen Hines, Stilwell, KS (US)

Correspondence Address: Frank B. Flink, Jr, Esq.;Griffin, Flink, and Watson, LLC, 8347 Fontana, Prairie Village, KS, 66207, US

(21) Appl. No.: 11/366,082

(22) Filed: Mar. 2, 2006

Related U.S. Application Data
(60) Provisional application No. 60/658,068, filed on Mar. 3, 2005.

(51) Int. Cl. A61F 2/06

(52) U.S. Cl. 623/1.49

(57) ABSTRACT

The present invention is directed to an intravascular treatment for intracranial aneurysms. The invention consists of a patch stent and, also, a patch stent delivery system allowing one to rotate the stent to align the patch with the neck of the aneurysm. A self-expanding nitinol framework holds the patch in place, a radiopaque agent allows the patch location to be visualized and a pusher tube that is mechanically locked into the unexpanded stent is used to push the stent out of the catheter with rotational and longitudinal control necessary to align the patch with the aneurysm. A self-expanding framework is used to support a patch at the neck of the aneurysm. The patch is designed to reduce the blood circulation in the aneurysm and the stagnant blood will clot or thrombus. The thrombus will stop any current blood leakage into the brain and will dramatically reduce the possibility of future leaks or potentially deadly ruptures. Over time the thrombus will be absorbed and the volume of the aneurysm will shrink reducing pressure on surrounding tissue.

### CROSS-REFERENCE TO RELATED APPLICATIONS

This application claims priority based upon provisional application 60/658,068 filed Mar. 3, 2005.

### STATEMENT REGARDING FEDERALLY SPONSORED RESEARCH OR DEVELOPMENT

Not Applicable.

### COPYRIGHT NOTICE

Portions of the disclosure, including the figures, contain material which is subject to copyright protection. The copyright owner has no objection to the facsimile reproduction by anyone of the patent document or the patent disclosure, as it appears in the Patent and Trademark Office patent file or records, but otherwise reserves all rights whatsoever.

### TECHNICAL FIELD

The present invention is directed to the field of medical and veterinary endovascular treatments of aneurysms and, more particularly, treatment of neurovascular aneurysms.

### BACKGROUND OF THE INVENTION

Neurovascular aneurysms are currently treated by two methods. The original treatment is an open surgical procedure called clipping that removes the aneurysm from the circulatory system by placing a clip at the base of the aneurysm. A newer, less invasive, endovascular procedure called coiling entails packing the aneurysm with flexible platinum coils that reduce blood circulation in the aneurysm and, thereby, triggering a thrombus in the aneurysm that may stop blood leakage and reduce the threat of the aneurysm bursting. Self-expanding nitinol stents are sometimes used with coiling. The stents form a lattice over the entrance to the aneurysm from the blood vessel, called the neck of the aneurysm, to help keep coils from prolapsing into the parent artery.

An aneurysm is formed when a weak spot in an artery stretches so thin that it is in danger of bursting from the pressure of the blood it contains. It forms a bulge or a ballooning area that may leak or rupture. An aneurysm that ruptures in a brain artery causes a stroke. Aneurysms that have wide openings at their base are called “wide neck” aneurisms and are the most difficult to treat. Wide neck aneurysms generally are defined as having a neck >4 mm or a dome-to-neck ratio <2.

About 5 million people in the United States currently have a brain aneurysm, and about 25 percent of these are “wide neck” aneurysms. In the United States it is estimated that as many as 18 million people will develop a brain aneurysm during their lifetime. Every year it is estimated that more than 30,000 people suffer from ruptured brain aneurysms. Ten to 15 percent of these patients will die before reaching the hospital. More than 50 percent will die within the first 30 days after rupture. Of those who survive, approximately half suffer some permanent neurological deficit.

An aneurysm may cause pain from pressure on surrounding organs, but often aneurysms have no symptoms. Aneurysms may be discovered during routine medical exams or diagnostic procedures for other health problems, but most often people are unaware of a problem until a rupture occurs. As relatively simple, viable treatments for aneurysms are developed physicians will look for and find more silent aneurysms and treat them before they cause problems.

The potential benefits of aneurysm treatments by clipping or coiling often do not outweigh the risks, especially for patients in whom remaining life expectancy is less than 20 years (those over age 60).

Neurosurgical clipping involves a craniotomy, an invasive, open surgical procedure with high risk. During this procedure, the arteries are exposed and one or more clips are applied across the neck of the aneurysm to stop blood from flowing into the aneurysm. The risk of a craniotomy is exacerbated in patients with a recent brain injury as well as in elderly or medically complicated patients. There is potential for further injury to the brain and additional neurological defect.

Endovascular coiling is a less invasive, non-surgical technique that involves inserting detachable platinum coils via a catheter into the aneurysm. The goal of endovascular coiling is to tightly pack coils inside the aneurysm to restrict blood flow within the aneurysm, and thus form a thrombus. The formation of a thrombus leaves little or no liquid in the aneurysm, eliminating the potential for the aneurysm to expand, leak or burst. The use of platinum allows the coils to be visible via X-ray. Although the endovascular coiling process plays a role in the treatment of brain aneurysms, the process has limitations. When platinum coils fill the aneurysm, the aneurysm size will remain basically the same and, therefore, it will continue to interfere with surrounding tissue. The coiling procedure requires a long learning process due to its technical difficulty. The process is effective in only a small percentage of aneurysms, such as the small neck aneurysms where the coils are more likely to stay in place. In other aneurysms, the coils are likely to protrude into the parent vessel with risk of clot formation and embolism.

Physicians have begun using stents or balloon-stent combinations in combination with coiling to improve the effectiveness of coiling. A balloon may sometimes be used to push the coils into or pack them into the aneurysm. With stent-assisted coiling, a stent is used to line the artery and form a screen to hold the platinum coils inside the aneurysm.

For direct treatment of neurovascular aneurysms, today's balloon-expandable or self-expanding stent designs are inadequate. Substantial open spaces in the walls of self-expanding stents and balloon-expandable stents do not sufficiently cover the aneurysm to block blood flow to the aneurysm. For example, in the stent-assisted coiling procedure, physicians currently use a thin self-expanding stent developed by the Boston Scientific Corporation. This product was approved for use by the FDA in 2002 for use with coils for the treatment of wide neck, intracranial, saccular aneurysms arising from a parent vessel with a diameter of ≧2 mm and ≦4.5 mm that are not amenable to treatment with surgical clipping. The flexibility of this Boston Scientific stent is derived from its very open design. It is intended to keep the coils in place, but the surface has a significant amount of open space and is not intended to block blood circulation across the neck of the aneurysm.

A stent with a greater percent solid area would restrict blood circulation into the aneurysm and trigger a thrombus in the aneurysm more effectively. In that event, the liquid aneurysm would solidify, eliminating the danger of rupture or leakage. If the aneurysm is filled with the thrombus only and no coils, the aneurysm sack will shrink as the thrombus is absorbed, reducing pressure on the surrounding tissue.

Stents are generally designed as cylindrical shells comprised of interconnected elements or struts. The pattern of struts on the surface of the cylinder allows a stent to be crimped to a small diameter for delivery and to expand radially from the small delivery diameter to a larger placement diameter once positioned within the lumen. The final placement diameter of an expandable stent is generally between 2.5 and 4 times the delivery diameter. As a result, the surface of the expanded stent has a significant amount of open space. At the small delivery diameter, the metal struts of the stents cover about 50 percent of the surface area of the stent. At the expanded placement diameter, the area covered by the struts is only about 12 to 20 percent of the stent wall. Current research indicates that a dense stent will reduce flow into the aneurysm. The open area of a typical stent, then, is a limitation with respect to treatment of an aneurysm.

Several additional types of stents and methods for making stents have been described previously. For example, the documents U.S. Pat. Nos. 6,080,191, 6,007,573, and 6,669,719 discuss stents using methods involving rolled flat sheets. U.S. Pat. No. 6,361,588 discusses a helical stent that expands into a relaxed helical shape when released from a catheter. U.S. Pat. No. 6,689,159 discusses a radially expandable stent with cylindrical elements and where expansion occurs when the stress of compression is removed. U.S. Pat No. 6,723,119 discusses a stent that is longitudinally expandable before and after expansion. These stents are a self-expanding type that expand into a cylindrical shape. A bifurcated stent design is discussed in U.S. Pat. No. 6,706,062 and U.S. Pat. No. 6,770,091 (the '062 and '091 patents) in which two portions of the stent are balloon expanded with two balloon catheters or separate pressures. Each branch of the stent is expanded once with a balloon.

Additional methods for treating aneurysms have been suggested. For example, the document U.S. Pat. No. 6,569,190 discusses a method for treating aneurysms that involves filling an aneurysmal sac with a non-particulate agent or fluid that solidifies in situ. This process leaves a permanent lump cast in the volume of the aneurysm. The lump is an undesirable side effect of solidification of the aneurysm volume.

U.S. Pat. No. 6,056,767, SYSTEM FOR THE TREATMENT OF A BODY DUCT AND PROCESS FOR ITS MANUFACTURE, describes a stent and delivery system that provide a stent that could be placed against the neck of an aneurysm but no provision for selective coverage of just the neck limits its usefulness to relative straight sections of arteries with no perforator (side branch arteries) that would be blocked by the stent.

The pleated stent assembly of U.S. patent application Ser. No. 10/695,527 filed on Oct. 28, 2003(the '527 Application) describes a stent for endovascular treatments that has advantages over other methods of treating aneurysms, in that, among other things, it provides a relatively solid area for closing off the aneurismal sac. However, the pleated stent of '527 application, since it is solid over the full stent cylinder, is limited in that it can not be used for aneurysms near side branch or perforator arteries that would also be occulted by the stent. The micro-pleated stent assembly of U.S. patent application Ser. No 11/031,899 filed on Jan. 7, 2005 (the '899 Application) describes a stent for endovascular treatments of aneurysms that may be patterned with a patch to block the neck of the aneurysm and avoid any near by perforators. The stents of both '527 and '899 are balloon expandable and made from a ductile material. Being constructed from a ductile material limits their use to inside 10 the skull where the stents will not be crushed by external forces. The ductile material must also be thick enough to be sufficiently rigid to withstand vasospasms that could also deform the ductile stent that has no capability to spring back. The necessity for the stents to be thick and the need to use a balloon for delivery thus limits the use of the stents of both the '527 and the '899 applications.

Thus, a number of limitations exist in the existing technology for treatment of aneurysms. The risk associated with open surgery often outweighs the potential benefits. Coiling is limited to narrow neck aneurysms and is a technically challenging procedure requiring poking a guidewire and many, often over 20 coils into the sack of a fragile aneurysm. Coils can prolapse into the parent artery causing a life-threatening thrombus to form. Stents of the '527 and '899 applications are limited to intracranial applications and have limited deliverability.

### BRIEF SUMMARY OF THE INVENTION

The current invention consists of a self-expanding patch stent and delivery system. The stent is delivered endovascularly, to cover the neck of an aneurysm and start a healing process that eliminates the dangers of the aneurysm. A self-expanding, preferably nitinol, framework holds the patch in place, and a radiopaque agent allows the patch location relative to the aneurysm to be visualized with standard x-ray angiography. A pusher tube, engaged with the stent, is used to push the stent out of a microcatheter with the rotational and longitudinal control necessary to align the patch with the aneurysm. The patch and framework, being self-expanding, will, upon release from the catheter, flex to expand to a shape dictated by the original shaping of the material. Thus, the self-expansion property is a feature of the elasticity of the material utilized for the patch and framework.

The preferable components of the patch stent are (1) the patch, which is a generally circular or elliptical disc shape and slightly larger than the neck of the aneurysm, and (2) a self-expanding framework to hold the patch in place over the neck of an aneurysm. The patch stent preferably contains radiopaque elements to visualize the patch location.

The preferred patch stent delivery system consists of a catheter, through which the stent, guidewire and pusher tube pass. The distal end of the pusher tube has radial, outward protruding fingers that engage the collapsed stent in the catheter. When engaged the pusher tube allows the individual placing the stent to control the longitudinal rotational position of the stent relative to the outer catheter tube. When properly oriented the stent is pushed out while the tube is pulled back. As the stent/finger engagement point moves beyond the end of the catheter, the stent expands to anchor into the artery and disengage from the fingers. The pusher tube, guidewire and catheter are then removed. Until the fingers disengage, the stent can be pulled back into the catheter and relocated if necessary.

The patch preferably is a mesh-structure which is sufficiently solid to reduce circulation in the aneurysm and to trigger a thrombus. The patch solid area will typically be greater than 50 percent. The patch may be constructed from metal, plastic, or combinations of the two. Open areas in the patch structure will improve the patch's flexibility to facilitate deliverability of the stent and small open areas will also facilitate endothelialization. The patch also may be constructed from nitinol, polytetrafluoroethylene, silicone, polyester or any other biocompatible material. The patch may consist of one or two thin disks bonded to a nitinol framework. The framework may be sandwiched between two disks. The patch material may be a continuous film, a porous film, a woven fabric, or a fibrous mat.

To allow the stent to be visualized angiographically, the patch location preferably will be indicated by materials with high radiopacity. This preferably would be accomplished by (1) pattern electroforming bands of gold or other highly radiopaque materials around selected nitinol struts, (2) construction of the patch from a thin layer or layers of a highly radiopaque materials, for example, gold or platinum, (3) coating or incorporating a dissolvable layer of radiopaque material, for example, iodine or iodixanol, into the patch or (4) combination of these techniques.

The self-expanding patch framework preferably is constructed from superelastic material, such as nitinol. The framework may be constructed from a tube or a flat sheet. If constructed from a tube, the cylindrical photolithography process taught in U.S. Pat. No. 6,274,294(the '294 patent), CYLINDRICAL PHOTOLITHOGRAPHY EXPOSURE PROCESS AND APPARATUS by Hines may be used to pattern a thin walled tube. The tube may be formed by conventional drawing techniques or may be formed by thin film sputter deposition. Sputtered thin film nitinol would require heat treatment to convert the amorphous as-deposited material to a crystalline material with the desired superelastic properties. The pattern of the support rings may be chosen to be compatible with forming from a flat sheet or a less restrictive cylindrical photolithography method may be used. Conventional 2D photolithography or cylindrical photolithography would be followed by electro-etching or chemical machining to remove the unwanted material, forming the framework. Laser machining or conventional machining could also be used to cut the stent pattern.

The framework would ultimately take the form of a pattern on the surface of a cylinder. If built by removing material from the wall of a tube, this would be the natural outcome. If the framework pattern is formed from sheet stock, the pattern must then be held against the surface of a cylindrical mandrel while being heat-treated to lock in the shape. Typical nitinol compositions would be annealed at about 500 degrees C. to obtain good superelastic properties and transform the patterned sheet material into a pattern cylindrical stent framework.

The framework would be very light and open to minimize the potential to block a perforator artery. Perforators are side branch arteries that may be found in the vicinity of an aneurysm.

The framework may consist of a screen or lattice network or ring to support the thin patch material and one or more springy sinuous bands to anchor the patch stent to the parent artery. The light open structure would also facilitate folding or pleating the device to fit into a catheter for delivery. The self-expanding nature of the stent would allow it to recover from vasospasms, common with aneurysm treatments. The stents could also be placed in arteries outside of the skeletal framework where it would be subject to external forces that could temporarily deform the artery and stent.

The patch location preferably should be visible radiographically to guide the patch's placement. A conventional guidewire would be used to guide the catheter to a location just proximal to the aneurysm. The guidewire could be located in the lumen of the pusher tube and either threaded through or alongside the stent. Alternatively, the guidewire could be removed after the microcatheter is in position and the stent and stent pusher could be inserted in the proximal end of the microcatheter and advanced to the aneurysm.

The pusher tube will have one or two radial protruding fingers that just fit inside the microcatheter. The proximal end of the folded or pleated stent structure will engage the fingers, locking the fingers to the stent as long as the proximal end of the stent is in the catheter. Locking the fingers to the stent allows the stent to be rotated and pulled back into the catheter if necessary. Locking provides rotational and longitudinal control of the patch stent and, thus, allows the patch to be located over the neck of the aneurysm. During patch placement, with the patch partially extending beyond the catheter and partially expanded, the angular position of the patch relative to the neck of the aneurysm can be viewed radiographically and adjusted to align the patch. When aligned, the catheter is pulled back while the pusher is adjusted to hold the patch in place with respect to the aneurysm. Only when the framework is free from the catheter, and thus free to expand, do the fingers locking the stent disengage. Once the fingers locking the stent are disengaged, the pusher may be used to hold the stent relative to the aneurysm while the microcatheter is pulled back to free the stent. The catheter and pusher may then be removed leaving the stent in place with the support rings anchored into the parent artery.

In another embodiment, the patch would be guided into place by the guidewire, rather than using the fingers on the pusher. In this embodiment, the guidewire engages the patch directly.

### BRIEF DESCRIPTION OF THE DRAWINGS

FIG. 1 shows 2-dimensional patch stent prior to being formed on to the surface of a cylinder. FIG. 2 shows a cross-section of an artery with a patch stent deployed at an aneurysm. FIG. 3A shows a transverse cross-section of a strut of the superelastic framework over-plated with a radiopaque material. FIG. 3B shows a longitudinal cross-section of a strut of the superelastic framework over-plated with a radiopaque material. FIG. 4 shows a cross-section of an artery and aneurysm with stent and delivery catheter.

### DETAILED DESCRIPTION OF PREFERRED EMBODIMENT

In the preferred embodiment the stent framework is made of superelastic nitinol. The patch is a continuation to the framework but with a high percent solid area. The location of the patch is made visible by an electroplated gold layer placed over selected sections of the framework.

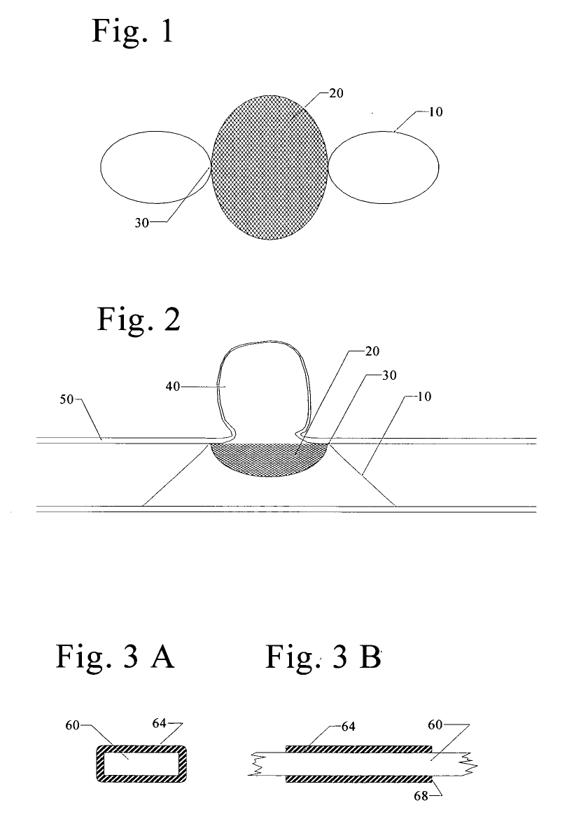

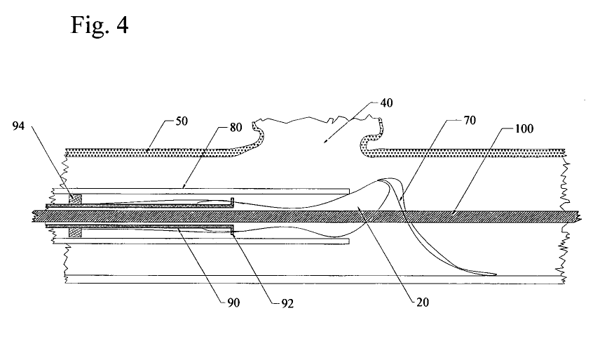

The framework preferably would be patterned from a 2-dimentional sheet of nitinol with a thickness between 10 and 50 microns. The sheet may be formed on a copper substrate by sputtering from an appropriately alloyed nitinol target (approximately 50 weight percent Ti and 50 weight percent Ni). The sheet of nitinol is patterned as shown in FIG. 1 to a pattern consisting of two elliptical rings 10 on opposite ends of a central patch 20 connected to a flexible area 30. The minor axis of the elliptical rings is slightly larger than the diameter of the artery 50 that will receive the stent. Artery diameters will typically be between 2 and 4 mm. The major axis of the elliptical rings will be the square root of 2 times the artery diameter to provide a patch support that will lay in the artery at an angle of about 45 degrees. The central patch is designed to be slightly larger (2 to 4 mm) than the neck of the aneurysm. This results in a patch which is generally an elliptically-shaped disc with minor and major axes of up to 6 and 10 mm, respectively. The patch solid area will be between 30 and 100 percent solid with about 70 percent being typical. Standard 2-dimentional photolithography defines the pattern and electro-etching is used to remove the unwanted nitinol. The copper substrate is then dissolved creating the framework shown in FIG. 1.

The framework as shown in FIG. 1 is coated with liquid resist. The resist may be sprayed on or dip coated. If dip coated, the excess may be removed by spinning so that centrifugal force slings off most of the resist leaving a thin coating. After a soft-bake, the resist is imaged from both sides to define the area on the nitinol struts for gold electroplating. As shown in FIG. 3A, areas of the nitinol strut 60 are gold plated to form bands 64 with the edge of the bands 68 defined by photoresist. The gold bands 64 are located in areas of the nitinol struts designed for minimum bending so that the gold added for radiopacity does no significantly reduce the elastic properties of the framework.

Preferably, the flat framework is forced to conform to the surface of a cylindrical ceramic mandrel after threading the mandrel through the elliptical rings 10. The rings are bent at the flexing point 30 at an angle of about 45 degrees. An outer split cylindrical mold is used to force the patch to the cylindrical surface of the ceramic mandrel. The cylindrical mandrel has a diameter slightly larger than the artery 50, where the stent will be deployed. The nitinol/gold stent is then heat treated at about 500 degrees C. to set the memory of the superelastic nitinol framework to the cylindrical shape.

FIG. 4 shows the distal end of the delivery catheter with the stent practically deployed. To deploy the stent, the guidewire 100 would be advanced beyond the aneurysm. The guidewire 100 would typically be a standard 0.012 inch wire. The pusher tube 90 and stent 70 are threaded over the guidewire 100 and the patch 20 of stent 70 is rolled around the pusher 90 and engaged with fingers 92. The elliptical rings 10 are elongated to fit into the catheter tube 80. Catheter tube 80 keeps the stent 70 locked to the fingers until the tube is pulled proximally and/or the pusher and stent are advanced distally until the stent springs away from the fingers. While the stent 70 and the pusher tube fingers 92 are engaged, the rotation of the proximal end of the pusher will rotate the patch 20. With the patch 20 partially extended beyond the catheter 80 and partially expanded, as shown in FIG. 4, the patch location can be visualized angiographically relative to the aneurysm 40 and the patch can be aligned with the aneurysm 40 before the stent is released for the fingers 92. Pusher ring 94 prevents the stent from moving relative to the aneurysm as the microcatheter tube 80 is pulled back to free the proximal elliptical support ring 10 by pushing against the proximal ring. With the stent in position, all hardware except the stent is removed.

In another embodiment, the patch is guided into place by the guidewire, rather than using the fingers on the pusher. In this embodiment, the guidewire is threaded through the pusher tube and through the proximal support ring and through a hole in the center of the patch. The guidewire would be internal to the support ring on the proximal end and external to the patch and support ring distal to the center of the patch. The flexible thin patch will allow the guidewire to lie straight in the catheter with the patch deforming to accommodate the straight guidewire. A sandwich polytetrafluoroethylene patch would reduce friction allowing the guidewire to be advanced freely. The tip of the guidewire would be advanced into the aneurysm prior to pushing the stent from the catheter. The guidewire will tend to align the patch over the neck of the aneurysm. Guidewire alignment may be used in conjunction with the locked pusher/stent and radiopaque markers to aid manual alignment or may be used in place of manual stent rotation to automatically rotate and guide the patch into position. The guidewire alignment may be particularly useful for locating a patch stent at a bifurcation. The stent could be delivered with only one end anchored in one side of the bifurcation. With one end anchored and the guidewire in the aneurysm helping to hold the patch in place, a second wire with an appropriate distal bend would be used to nudge the free end of the stent into the other side of the bifurcation.

In the above embodiments, the patch is rolled around the central pusher tube, which pusher tube is 0.028 inches in diameter, and the catheter tube 80 is 4 French, or about 0.052 inches in diameter. To reach distal aneurysms, a smaller catheter will be needed. A 2 French microcatheter and a 0.014 inch pusher tube can be used to obtain additional flexibility for reaching more distal aneurysms but the volume between the pusher tube and the catheter tube is insufficient to accommodate simply rolling the patch around the pusher tube. Therefore, an additional embodiment accommodates smaller diameters by patterning the patch so that it may be stretched in addition to being rolled around the pusher tube, thereby providing a greater volume to store the stent in while it is in the catheter. However, a stretched stent may not readily be deployed by simply pushing the elongated stent from the catheter, because pushing a stretched stent will tend to make it bunch up in the catheter. Therefore, in this alternate embodiment, the stent may need to be pulled from the catheter. To pull the stent from the catheter tube, a forked guidewire with forks on the distal end may be used. The forks on the guidewire are slanted towards the distal end of the catheter, so that the fork can engage the stent selectively, i.e., engage on a push of the forked guidewire towards the distal end of the catheter and not engage on a pull of the forked guidewire towards the proximal end of the catheter. The non-forked guidewire 100 in FIG. 4 preferably will be used to initially guide the catheter with the patch stent into place. Once in place, the non-forked guidewire is pulled proximally from the catheter, and the forked guidewire inserted into the catheter. As the distal forked tip of the forked guidewire emerges from the pusher tube, the fork springs apart and catches in the struts of the stent. Advancing the forked guidewire will tend to pull the stent from the catheter tube. To deploy the stent, pushing and pulling of the forked guidewire will both be used. As the fork nears the end of the catheter, as determined by a gauge on the proximal end of the catheter, the forked guidewire is pulled back to get a new bite on the stent. Advancing the forked guidewire again will tend to pull the stent from the catheter tube further. The forked guidewire is thus pushed and pulled until the stent is fully deployed. When the patch is partially deployed and therefore visible radiographically, the forked guidewire may be used to rotate the patch into position. In this embodiment, it may be preferable to eliminate the fingers on the pusher tube, to avoid the need to coordinate the actions of the fingers and forks on the stent as it is deployed.

Minor variations on the delivery method will allow the stent to be used to treat aneurysms at bifurcations. To treat an aneurysm at a bifurcation, the distal elliptical ring will be deployed into one arm of the bifurcation and, after the stent patch is aligned with the neck of the aneurysm, the second ring could be nudged into the second arm of the bifurcation with a special pusher guidewire bent at the distal tip and designed to catch on the support ring.

As noted in the Brief Summary of the Invention, many variations of the preferred embodiments are possible. The stent may be formed from cylindrical stock instead of sheet stock to provide more pattern options. The high percent solid area of the patch may be obtained by using a plastic or gold disk with appropriate fenestrations. Radiopacity may be obtained through the use of a biodegradable or dissolvable material like iodine or iodixanol. Superelastic tubes may be nitinol or other alloys. Nitinol may be wrought or vacuum deposited. Heat treat parameters will be a function of the nitinol alloy. Stent, catheter and associated hardware sizes will be adjusted to be appropriate for the arteries where the stents are to be used. The stent pattern and the patch pattern may be varied extensively.

What is claimed and desired to be secured by Letters Patent is as follows:

1. A vascular stent for treating blood vessel defects comprising:
a patch, configured to cover a neck of an aneurysm and
a support framework configured to anchor the stent in an artery.

2. The stent as claimed in claim 1 further comprising
a radiopaque agent applied to the stent.

3. The stent as claimed in claim 2 wherein said patch is a disc shape, said patch is self-expanding, said patch having proximal and distal ends, and wherein said framework consists of a first and second loop, the first said loop flexibly attached to the proximal end of said patch and the second said loop flexibly attached to the distal end of said patch.

4. The stent as claimed in claim 2 wherein said patch is a material selected from the group of metal, plastic, or a combination of metal and plastic, and wherein said patch is self-expanding.

5. The stent as claimed in claim 3 wherein said patch disc shape is elliptical in configuration, wherein the elliptical shape has major and minor axis dimensions of less than or equal to ten and six millimeters, respectively.

6. The stent as claimed in claim 2 wherein said patch is a material selected from the group of nitinol, polytetrafluoroethylene, silicone, and polyester.

7. The stent as claimed in claim 3 wherein said patch is formed of a mesh material, which mesh material has an open area less than 70 percent.

8. The stent as claimed in claim 3 wherein said patch is formed of a mesh material, which mesh material has an open area less than 50 percent.

9. The stent as claimed in claim 1 wherein said patch is comprised of a first and second disk, said support framework comprised of nitinol configured to have a first disc and second loop and a center portion between said loops, and wherein said center portion is sandwiched between said first and second disks.

10. The stent as claimed in claim 9 wherein said patch is a material selected from the group of a continuous film, a porous film, a woven fabric, or a fibrous mat.

11. The stent of claim 3 further comprising a stent delivery system, said delivery system comprising a catheter and a pusher, wherein said catheter is a flexible tube defining a proximal end, a central opening, and a distal end and wherein, prior to deployment of said stent, said stent is located within said central opening defined by said catheter, and said pusher has fingers which fingers releasably engage said stent thereby providing longitudinal and rotational control of said stent and wherein said engagement releases when said stent expands beyond the distal end of said catheter.

12. The stent as claimed in claim 11 wherein said pusher is a flexible tube extending through the central opening defined by said catheter, said pusher defining a central opening, and further comprising a guidewire extending through the central opening defined by said pusher.

13. The stent of claim 3 further comprising a stent delivery system, said delivery system comprising a catheter, a pusher, and a guidewire wherein said catheter is a flexible tube defining a proximal end, a central opening, and a distal end and wherein, prior to deployment of said stent, said stent is located within said central opening defined by said catheter, and wherein said pusher is a flexible tube extending through the central opening defined by said catheter, said pusher defining a central opening, and wherein said guidewire extends through the central opening defined by said pusher, and wherein said guidewire further comprises a fork, which fork is configured to releasably engage said stent and thereby pull said stent towards said distal end of said catheter, which fork releases said stent when said guidewire is moved towards the proximal end of said catheter.

14. A vascular stent for treating blood vessel defects comprising:
a patch and a framework, wherein said patch is sufficiently solid to reduce circulation into an aneurysm and said patch is configured to be conformed to a portion of a blood vessel, and wherein said framework is flexibly attached to said patch and said framework is configured to support said patch against a wall of the blood vessel.

15. The stent as claimed in claim 14 further comprising:
a radiopaque agent applied to the stent.

16. The stent as claimed in claim 15 wherein said patch is a disc shape which is generally elliptical in configuration, said patch having proximal and distal ends; and wherein said framework consists of a first and second loop, the first said loop flexibly attached to the proximal end of said patch and the second said loop attached to the distal end of said patch.

17. The stent as claimed in claim 16 wherein said stent consists essentially of nitinol.

18. The stent of claim 14 further comprising a stent delivery system, said delivery system comprising a catheter and a pusher, wherein said catheter is a flexible tube defining a proximal end, a central opening, and a distal end and wherein, prior to deployment of said stent, said stent is located within said central opening defined by said catheter, and said pusher releasably engages said stent thereby providing longitudinal and rotational control of said stent and wherein said engagement automatically releases when said stent expands beyond the distal end of the catheter.

19. The stent of claim 14 further comprising a stent delivery system, said delivery system comprising a catheter, a pusher, and a guidewire wherein said catheter is a flexible tube defining a proximal end, a central opening, and a distal end and wherein, prior to deployment of said stent, said stent is located within said central opening defined by said catheter, and wherein said pusher is a flexible tube extending through the central opening defined by said catheter, said pusher defining a central opening, and wherein said guidewire extends through the central opening defined by said pusher, and wherein said guidewire further comprises a fork, which fork is configured to releasably engage said stent and thereby pull said stent towards said distal end of said catheter, which fork releases said stent when said guidewire is moved towards the proximal end of said catheter.

20. The stent of claim 16 further comprising a stent delivery system, said delivery system comprising a catheter and a pusher, wherein said catheter is a flexible tube defining a proximal end, a central opening, and a distal end and wherein, prior to deployment of said stent, said stent is located within said central opening defined by said catheter, and said pusher releasably engages said stent thereby providing longitudinal and rotational control of said stent and wherein said engagement automatically releases when said stent expands beyond the distal end of the catheter.

21. The stent of claim 16 further comprising a stent delivery system, said delivery system comprising a catheter, a pusher, and a guidewire wherein said catheter is a flexible tube defining a proximal end, a central opening, and a distal end and wherein, prior to deployment of said stent, said stent is located within said central opening defined by said catheter, and wherein said pusher is a flexible tube extending through the central opening defined by said catheter, said pusher defining a central opening, and wherein said guidewire extends through the central opening defined by said pusher, and wherein said guidewire further comprises a fork, which fork is configured to releasably engage said stent and thereby pull said stent towards said distal end of said catheter, which fork releases said stent when said guidewire is moved towards the proximal end of said catheter.

22. A vascular stent for treating blood vessel aneurysms comprising:
a patch, a support framework, and a radiopaque agent;
wherein said patch is disc-shaped and configured to fit across a neck opening of an aneurysm, said patch disc diameter up to 4 millimeters larger than the aneurysm neck opening, said patch having proximal and distal ends; and
wherein said framework consists of a first and second loop, the first said loop flexibly attached to the proximal end of said patch and the second said loop attached to the distal end of said patch, said loops configured to fixedly engage blood vessel walls and hold said patch against the aneurysm neck, and wherein said patch and frame consist essentially of super-elastic material so as to be self-expanding.

23. The stent as claimed in claim 22 wherein said patch disc shape is elliptical in configuration, wherein the elliptical shape has major and minor axis dimensions of less than or equal to ten and six millimeters, respectively.

24. The stent of claim 22 further comprising a stent delivery system, said delivery system consisting of a catheter and a pusher, wherein said catheter is a flexible tube defining a proximal end, a central opening, and a distal end and wherein, prior to deployment of said stent, said stent is located within said central opening defined by said catheter, and said pusher releasably engages said stent thereby providing longitudinal and rotational control of said stent and wherein said engagement automatically releases when said stent expands beyond the distal end of the catheter.

25. The stent of claim 22 further comprising a stent delivery system, said delivery system comprising a catheter, a pusher, and a guidewire wherein said catheter is a flexible tube defining a proximal end, a central opening, and a distal end and wherein, prior to deployment of said stent, said stent is located within said central opening defined by said catheter, and wherein said pusher is a flexible tube extending through the central opening defined by said catheter, said pusher defining a central opening, and wherein said guidewire extends through the central opening defined by said pusher, and wherein said guidewire further comprises a fork, which fork is configured to releasably engage said stent and thereby pull said stent towards said distal end of said catheter, which fork releases said stent when said guidewire is moved towards the proximal end of said catheter.
